(19) United States
(12) Patent Application Publication
(10) Pub. No.: US 2021/0190680 A1
(43) Pub. Date: Jun. 24, 2021

(54) CANNABINOID DETECTION AND MEASURING DEVICE

(71) Applicants: Jacqueline Reyes, Fairfield, CT (US); Benjamin Warburton, Weehawken, NJ (US)

(72) Inventors: Jacqueline Reyes, Fairfield, CT (US); Benjamin Warburton, Weehawken, NJ (US)

(21) Appl. No.: 17/133,509

(22) Filed: Dec. 23, 2020

Related U.S. Application Data
(63) Continuation of application No. PCT/US2019/040078, filed on Jul. 1, 2019.
(60) Provisional application No. 62/692,144, filed on Jun. 29, 2018.

(51) Int. Cl. G01N 21/33; A61B 5/00

(52) U.S. Cl. CPC G01N 21/33; A61B 5/0082; A61B 5/0064; A61B 5/0004

(57) ABSTRACT

A cannabinoid measuring and detection device can include a power source, a UV light source operably connected to the power source and configured to output light in an output direction at an ultraviolet wavelength and at a known intensity, and a UV light sensor arranged in the output direction to detect the light output by the UV light source and output a detection signal corresponding to the detected light. The UV light source and UV light sensor can be arranged to enable body tissue of an individual to be placed between the UV light source and UV light sensor during a test period. At least one processor can be configured to receive the detection signal corresponding to the test period and determine whether a cannabinoid is present in the individual based on the known intensity of the output light and the detection signal.

### CROSS-REFERENCE TO RELATED APPLICATIONS

This application is a continuation of International Application No. PCT/US2019/040078, filed Jul. 1, 2019, which claims the benefit of U.S. Provisional Application No. 62/692,144, filed on Jun. 29, 2018. The disclosure of each of the above applications is incorporated herein by reference in its entirety.

### FIELD

The present disclosure relates to a cannabinoid detection and measuring device, and, more particularly, to a simple and effective cannabinoid detection and measuring device and method of use for detecting, measuring, etc. the presence of cannabinoids in the bloodstream of cannabis users that is non-invasive and provides near immediate results.

### BACKGROUND

As the cannabis industry grows and consumption of recreational and medicinal cannabis becomes more prevalent, the need for a simple and effective apparatus that can quickly measure the presence of cannabinoids in an individual's system is increasing. More specifically, an apparatus that can obtain a measurement in a non-invasive manner with an immediate reading will be necessary or at least highly desirable in the continued cannabis legalization process. Such an apparatus will be critical for law enforcement officials and others responsible for implementation/enforcement of the law, as well as for individuals, to ensure safety and to regulate personal consumption levels. There currently are no tools that provide immediate, accurate, non-invasive measurement of cannabinoids in an individual's system. In fact, existing methods of detection are widely variable and subjective in their testing results. While the cannabis industry has demonstrated a need for a device like this, most of the innovation in the industry has happened around farming, direct-to-consumer products, and distribution, and has not been directed to the measurement need discussed above.

### SUMMARY

The disclosed handheld cannabinoid measuring and detection device can be a handheld device that uses wavelengths of light passing through soft body tissue to measure the change in absorbance at specific wavelengths correlated to cannabinoids. This device is small, portable, and provides a near immediate and accurate reading.

In various embodiments of the present disclosure, a cannabinoid measuring and detection device is disclosed. The cannabinoid measuring and detection device can include a power source, a UV light source operably connected to the power source and configured to output light in an output direction at an ultraviolet wavelength and at a known intensity, and a UV light sensor arranged in the output direction to detect the light output by the UV light source and output a detection signal corresponding to the detected light. The cannabinoid measuring and detection device can further include at least one processor operably connected to the UV light sensor and the UV light source. The UV light source and UV light sensor can be arranged to enable body tissue of an individual to be placed between the UV light source and UV light sensor. The at least one processor can be configured to receive the detection signal corresponding to a test period during which the body tissue of the individual is placed between the UV light source and UV light sensor and determine whether a cannabinoid is present in the individual based on the known intensity of the output light and the detection signal.

In various aspects, the cannabinoid measuring and detection device can further include a first arm in which the UV light source is arranged, a second arm in which the UV light sensor is arranged, and a connecting portion coupling the first arm with the second arm. The connecting portion can enable adjustment of a position of the first and second arm with respect to each other. For example only, the connecting portion can comprise an adjustable hinge. Optionally, the connecting portion can enable adjustment of the position of the first and second arm while maintaining the UV light sensor in the output direction of the light output from UV light source.

In various aspects, the UV light source can output light in the ultraviolet A and ultraviolet B ranges. For example only, the UV light source can output light with wavelengths between 240 and 370 nanometers.

According to some implementations, during the test period, the at least one processor can determine whether the cannabinoid is present in the individual based on a difference between the known intensity of the output light and an intensity of the detected light represented by the detection signal. In such implementations, the difference between the known intensity of the output light and the intensity of the detected light represented by the detection signal can be related to absorbance of the output light by the body tissue of the individual.

According to some implementations, during the test period, the at least one processor can determine whether the cannabinoid is present in the individual based on a difference between the known intensity of the output light in the ultraviolet A and ultraviolet B ranges and an intensity of the detected light in the ultraviolet A and ultraviolet B ranges represented by the detection signal. Alternatively or additionally, during the test period, the at least one processor can determine whether the cannabinoid is present in the individual based on a difference between the known intensity of the output light with wavelengths between 240 and 370 nanometers and an intensity of the detected light with wavelengths between 240 and 370 nanometers represented by the detection signal.

In some aspects, the cannabinoid measuring and detection device can further comprise a detection component and an output component, wherein the detection component includes the UV light source and UV light sensor and the output component includes the at least one processor. In some cases, the detection component and the output component can be separate from each other. The detection component and the output component can be operably connected via a wired connection, via a wireless connection, or otherwise. The output component can further include a display configured to provide an output indicative of whether the cannabinoid is present in the individual. In some aspects, the cannabinoid measuring and detection device can further comprise an output device that is configured to provide an output indicative of whether the cannabinoid is present in the individual. In other aspects, the cannabinoid measuring and detection device can further comprise an output device that is configured to provide an output indicative of a level of cannabinoid present in the individual.

According to various additional embodiments of the present disclosure, a cannabinoid measuring and detection device is disclosed. The cannabinoid measuring and detection device can include a power source, a UV light source operably connected to the power source and configured to output light in an output direction at an ultraviolet wavelength and at a known intensity, and a UV light sensor arranged in the output direction to detect the light output by the UV light source and output a detection signal corresponding to the detected light. The cannabinoid measuring and detection device can further include at least one processor operably connected to the UV light sensor and the UV light source, a first arm in which the UV light source is arranged, a second arm in which the UV light sensor is arranged, and a connecting portion coupling the first arm with the second arm. The connecting portion can enable adjustment of a position of the first and second arm with respect to each other. The UV light source and UV light sensor can be arranged to enable body tissue of an individual to be placed between the UV light source and UV light sensor. The at least one processor can be configured to receive the detection signal corresponding to a test period during which the body tissue of the individual is placed between the UV light source and UV light sensor and determine whether a cannabinoid is present in the individual based on the known intensity of the output light and the detection signal. The UV light source can output light with wavelengths between 240 and 370 nanometers. During the test period, the at least one processor can determine whether the cannabinoid is present in the individual based on a difference between the known intensity of the output light and an intensity of the detected light represented by the detection signal.

In some aspects, the difference between the known intensity of the output light and the intensity of the detected light represented by the detection signal can be related to absorbance of the output light by the body tissue of the individual. Alternatively or additionally, the connecting portion can enable adjustment of the position of the first and second arm while maintaining the UV light sensor in the output direction of the light output from UV light source.

Further areas of applicability of the present disclosure will become apparent from the detailed description provided hereinafter. It should be understood that the detailed description and specific examples are intended for purposes of illustration only and are not intended to limit the scope of the disclosure.

### BRIEF DESCRIPTION OF THE DRAWINGS

The present disclosure will become more fully understood from the detailed description and the accompanying drawings, wherein:

FIG. 1 is a first perspective view of a detection component of an example cannabinoid measuring and detection device according to some aspects of the present disclosure;

FIG. 2 is a second perspective view of the detection component of FIG. 1;

FIG. 3 is a third perspective view of the detection component of FIG. 1; and

FIG. 4 is a perspective view of an output component of an example cannabinoid measuring and detection device according to some aspects of the present disclosure.

### DETAILED DESCRIPTION

As briefly mentioned above, the present disclosure relates to a simple and effective cannabinoid measuring and detection device that utilizes a non-invasive detection method. The disclosed cannabinoid measuring and detection device utilizes light, e.g., in the ultraviolet A (“UV-A”) and ultraviolet B (“UV-B”) spectra, which is passed through soft body tissue of an individual, and measures the change in absorbance at specific wavelengths correlated to cannabinoids. The light sensor can, for example, be a gallium nitride based sensor that is reactive to light in the UV-A and UV-B spectra, e.g., those with wavelengths between 240 and 370 nanometers. Between those wavelengths, it is known that cannabinoids exhibit characteristic absorption, which can be utilized to signal their presence in an individual, e.g., the bloodstream of the individual.

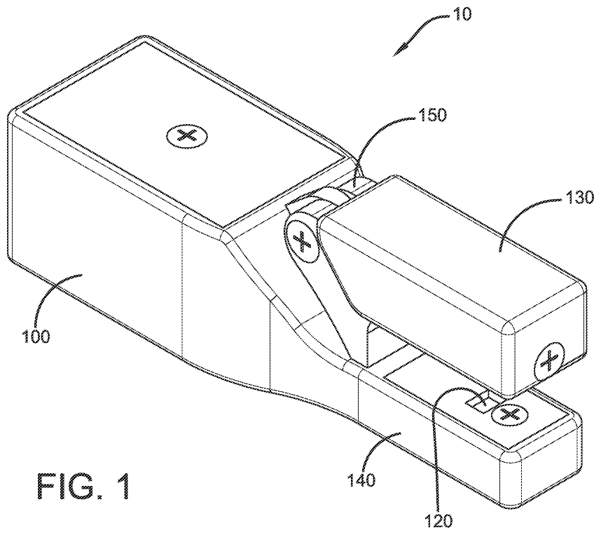

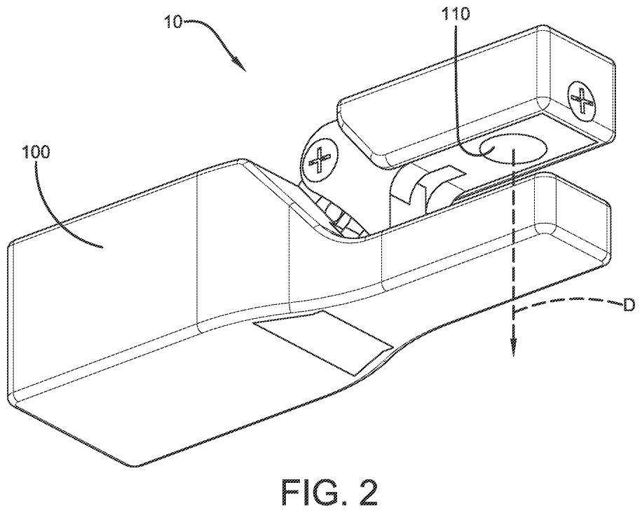

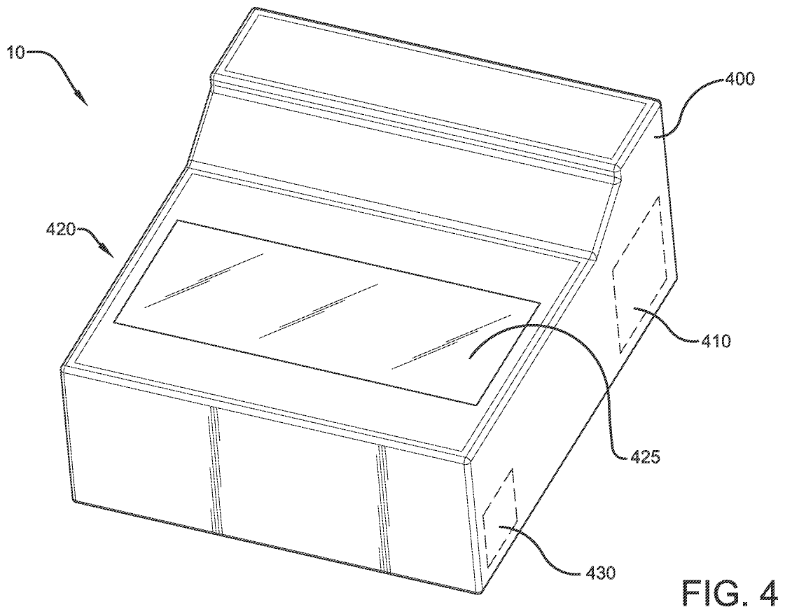

An example cannabinoid measuring and detection device 10 is illustrated in the attached figures. One skilled in the art will appreciate that the illustrated cannabinoid measuring and detection device 10 is merely an example and that modifications to the example device can be made without deviating from the scope of the present disclosure. The cannabinoid measuring and detection device 10 can include a detection component 100 (FIGS. 1-3) and an output component 400 (FIG. 4). Each of these components 100, 400 will be described in detail below.

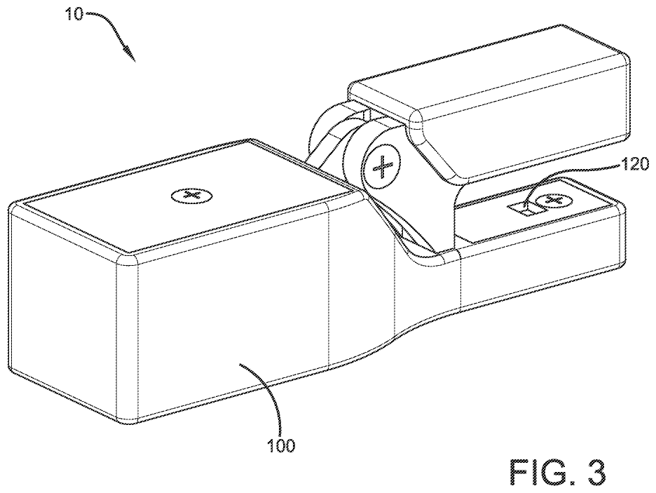

With reference to FIGS. 1-3, the detection component 100 can include an ultraviolet (“UV”) light source 110 and a UV light sensor 120. The UV light source 110 is operably connected to a power source (see, e.g., power source 430 in FIG. 4 described below). Further, the UV light source 110 is configured to output light in an output direction D (FIG. 3). The UV light sensor 120 can be arranged in a path of the output direction D such that the output light from UV light source 110 can be expected to be received by the UV light sensor 120. The UV light sensor 120 can be configured to output a detection signal corresponding to the light detected. Further, the light output by the UV light source 110 is at a UV wavelength and can be at a known intensity. In this manner, the cannabinoid measuring and detection device 10 can determine whether a cannabinoid is present in an individual, as more fully discussed below.

The output component 400 (FIG. 4) can include at least one processor 410 and at least one output device 420. Example output devices 420 include, but are not limited to, a display 425 (as illustrated), a speaker, and a haptic device. Other forms of output device(s) 420 are contemplated. The output device 420 is configured to provide an output indicative of whether the cannabinoid is present in the individual. Such outputs can include a binary output (cannabinoid detected v. absent) or a more precise output indicative of a level of cannabinoid present in the individual. The output component 400 can also include a power source 430, which can take the form of any known power source (battery, power adaptor and cord, etc.).

In the illustrated example, the detection component 100 and the output component 400 are separate from each other. The detection component 100 and output component 400 can be operably connected in various ways, such as via a wired connection (USB wire, etc.) and a wireless connection (Bluetooth, WiFi, Near Field Communication, etc.). Further, it should be appreciated that a cannabinoid measuring and detection device 10 having a combined detection component 100 and the output component 400 is within the scope of the present disclosure.

The example detection component 100 comprises a first arm 130 and a second arm 140 coupled together with a connecting portion 150, which is illustrated as an adjustable hinge. The first arm 130 can include the UV light source 110. The UV light source 110 can be any type of light source, for example, but not limited to, one or more light-emitting diodes (“LED(s)”). The UV light source 110 generates the light that will be passed through the body tissue of an individual, as further described below. In various aspects, the UV light source 110 can output light in the ultraviolet A and/or ultraviolet B ranges. For example only, the UV light source 110 can output light with wavelengths between 100 and 400 nanometers, between 240 and 370 nanometers, or any other acceptable range of wavelengths. It has been determined that cannabinoids exhibit characteristic absorption of light in the above recited ranges, which can be utilized to signal their presence in an individual, e.g., in an individual's bloodstream, as more fully described below.

The second arm can include UV light sensor 120, which can detect/measure the light passing through the body tissue of the individual. The connecting portion 150, such as the illustrated hinge, can enable adjustment of the position of the first and second arms 130, 140 with respect to each other, e.g., in order to adjust to body tissue of different sizes.

In some aspects, the connecting portion 150 can enable adjustment of the position of the first and second arms 130, 140 while maintaining the UV light sensor 120 in the output direction D of the light output from UV light source 110. In this manner, the UV light source 110 can be designed to transmit light substantially directly into the UV light sensor 120, e.g., with a maximum deflection of less than five (5) degrees. The use of LEDs as the UV light source 110 may further enable such directionality of the output light because LEDs are directional light sources. Accordingly, for a UV light source 110 that utilizes LED(s), it may be reasonably assumed that the majority of the light emitted from the UV light source 110 will reach the UV light sensor 120 unless it is absorbed or reflected. Thus, the light intensity output from the UV light source 110 can be approximately equal to the light received by the UV light sensor 120 if there is no absorption or reflection.

The cannabinoid measuring and detection device 10 is utilized to determine whether a cannabinoid is present in an individual based on the known intensity of the output light from UV light source 110 and the detection signal output by the UV light sensor 120. As mentioned above, the detection signal can correspond to the light detected by the UV light sensor 120. The at least one processor 410 is configured to receive the detection signal corresponding to a test period during which the body tissue of an individual is placed between the UV light source 110 and UV light sensor 120 in order to determine whether a cannabinoid is present in the individual.

During a test period, an individual can insert a portion of body tissue (e.g., soft tissue) between the UV light source 110 and the UV light sensor 120. An example of such body tissue is an individual's finger, although any appropriate tissue can be utilized. For example only, any body tissue that includes a blood vessel may be appropriate for the testing process. It should be appreciated, however, that body tissue that includes bone tissue may not yield the best results as bone can absorb light and possibly provide inaccurate results. In addition to a finger, further examples of appropriate body tissue include a nose, an ear, and a purlicue (the webbing between a thumb and forefinger). In order to reduce reflection from the skin surface, which may reduce the accuracy of the measurement of UV light absorption, the body tissue should be close to or in contact with both the UV light source 110 and UV light sensor 120. This may be enabled, for example, by the adjustment of the position of the first and second arms 130, 140 with respect to each other, as mentioned above.

In some implementations, an individual may initiate a test period by pressing a button or some other type of actuator. At that time, the UV light source 110 can begin outputting the light, e.g., in the UV ranges described above. Similarly, the UV light sensor 120 can begin detecting the light and output the detection signal. In some aspects, multiple detection signals can be output, combined, averaged, etc. from multiple measurements during one test period. In some aspects, the output light can be constant in presence and intensity during the course of the test period and the intensity of the light at the appropriate wavelengths is measured by the UV light sensor 120 and compared with the expected intensity for the output light. In other implementations, the output light can vary in presence, intensity, wavelength, etc. during a test period and one or multiple readings at the various different settings can be obtained. It should be appreciated that any form of obtaining the detecting signal is contemplated by the present disclosure.

Generally speaking, the more light (at the appropriate wavelengths) that is absorbed by the body tissue will correlate with a higher presence of cannabinoids in an individual. In some aspects, the at least one processor 410 determines whether the cannabinoid is present in an individual based on a difference between the known intensity of the output light and an intensity of the detected light represented by the detection signal. The difference between the known intensity of the output light (from UV light source 110) and the intensity of the detected light represented by the detection signal (output by the UV light sensor 120) can be related to absorbance of the output light by the body tissue of the individual. Further, in various implementations, during the test period, the at least one processor 410 can determine whether the cannabinoid is present in the individual based on a difference between the known intensity of the output light in the ultraviolet A and ultraviolet B ranges and an intensity of the detected light in the ultraviolet A and ultraviolet B ranges represented by the detection signal. In additional or alternative implementations, the at least one processor can determine whether the cannabinoid is present in the individual based on a difference between the known intensity of the output light with wavelengths between 240 and 370 nanometers and an intensity of the detected light with wavelengths between 240 and 370 nanometers represented by the detection signal.

In some examples, a logarithmic function can be utilized to assess the amount of light that is absorbed by the body tissue of an individual. The algorithm is a logarithmic function that tails to one (1), meaning that there is a known quanta of light at a known wavelength. This can be due to calibration of the UV light source 110, as well as the input power, and measured heat losses. The device 10 can be calibrated, e.g., by placing the UV light sensor 120 a relatively fixed distance away from the UV light source 110 and measuring the emission/detection. The percentage of light transmission through the skin for a body tissue of that same frequency is a known value, meaning that we can subtract the amount of light that the skin absorbs, and the remainder is the light that is potentially able to be absorbed by cannabinoids in the body tissue. The difference of the initial potential measurable light and the actual measured light can follow a logarithmic curve until all the light is accounted for, the emission, the percentage lost to skin contact, and ultimately what cannabinoids are present in the body tissue.

As mentioned above, the output device 420 is configured to provide an output indicative of whether the cannabinoid is present in the individual. This output can take various forms. For example only, the result can be output as a number (similar to a blood alcohol content number) that can be compared by an operator to an acceptable level of cannabinoids. In another example, the device can be set to correlate the result with a scale to output a binary output (cannabinoids present or not present, below maximum allowable level or above maximum allowable level, etc.). Furthermore, in some implementations, the device 10 can be calibrated to compensate for the body mass, medical conditions or medications, and/or other personal characteristics of the individual being tested, as well as ambient conditions during the test period (temperature, ambient light, etc.).

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known procedures, well-known device structures, and well-known technologies are not described in detail.

The terminology used herein is for the purpose of describing particular example embodiments only and is not intended to be limiting. As used herein, the singular forms “a,” “an,” and “the” may be intended to include the plural forms as well, unless the context clearly indicates otherwise. The term “and/or” includes any and all combinations of one or more of the associated listed items. The terms “comprises,” “comprising,” “including,” and “having,” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

As used herein, the term module may refer to, be part of, or include: an Application Specific Integrated Circuit (ASIC); an electronic circuit; a combinational logic circuit; a field programmable gate array (FPGA); a processor or a distributed network of processors (shared, dedicated, or grouped) and storage in networked clusters or datacenters that executes code or a process; other suitable components that provide the described functionality; or a combination of some or all of the above, such as in a system-on-chip. The term module may also include memory (shared, dedicated, or grouped) that stores code executed by the one or more processors.

Unless specifically stated otherwise as apparent from the above discussion, it is appreciated that throughout the description, discussions utilizing terms such as “processing” or “computing” or “calculating” or “determining” or “displaying” or the like, refer to the action and processes of a computer system, or similar electronic computing device, that manipulates and transforms data represented as physical (electronic) quantities within the computer system memories or registers or other such information storage, transmission or display devices.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

What is claimed is:

1. A cannabinoid measuring and detection device, comprising:
a power source;
a UV light source operably connected to the power source and configured to output light in an output direction at an ultraviolet wavelength and at a known intensity;
a UV light sensor arranged in the output direction to detect the light output by the UV light source and output a detection signal corresponding to the detected light; and
at least one processor operably connected to the UV light sensor and the UV light source,
wherein the UV light source and UV light sensor are arranged to enable body tissue of an individual to be placed between the UV light source and UV light sensor, and
wherein the at least one processor is configured to receive the detection signal corresponding to a test period during which the body tissue of the individual is placed between the UV light source and UV light sensor and determine whether a cannabinoid is present in the individual based on the known intensity of the output light and the detection signal.

2. The cannabinoid measuring and detection device of claim 1, further comprising:
a first arm in which the UV light source is arranged;
a second arm in which the UV light sensor is arranged; and
a connecting portion coupling the first arm with the second arm, the connecting portion enabling adjustment of a position of the first and second arm with respect to each other.

3. The cannabinoid measuring and detection device of claim 2, wherein the connecting portion comprises an adjustable hinge.

4. The cannabinoid measuring and detection device of claim 2, wherein the connecting portion enables adjustment of the position of the first and second arm while maintaining the UV light sensor in the output direction of the light output from UV light source.

5. The cannabinoid measuring and detection device of claim 1, wherein the UV light source outputs light in the ultraviolet A and ultraviolet B ranges.

6. The cannabinoid measuring and detection device of claim 1, wherein the UV light source outputs light with wavelengths between 240 and 370 nanometers.

7. The cannabinoid measuring and detection device of claim 1, wherein, during the test period, the at least one processor determines whether the cannabinoid is present in the individual based on a difference between the known intensity of the output light and an intensity of the detected light represented by the detection signal.

8. The cannabinoid measuring and detection device of claim 7, wherein the difference between the known intensity of the output light and the intensity of the detected light represented by the detection signal is related to absorbance of the output light by the body tissue of the individual.

9. The cannabinoid measuring and detection device of claim 1, wherein, during the test period, the at least one processor determines whether the cannabinoid is present in the individual based on a difference between the known intensity of the output light in the ultraviolet A and ultraviolet B ranges and an intensity of the detected light in the ultraviolet A and ultraviolet B ranges represented by the detection signal.

10. The cannabinoid measuring and detection device of claim 1, wherein, during the test period, the at least one processor determines whether the cannabinoid is present in the individual based on a difference between the known intensity of the output light with wavelengths between 240 and 370 nanometers and an intensity of the detected light with wavelengths between 240 and 370 nanometers represented by the detection signal.

11. The cannabinoid measuring and detection device of claim 1, further comprising a detection component and an output component, wherein the detection component includes the UV light source and UV light sensor and the output component includes the at least one processor.

12. The cannabinoid measuring and detection device of claim 11, wherein the detection component and the output component are separate from each other.

13. The cannabinoid measuring and detection device of claim 12, wherein the detection component and the output component are operably connected via a wired connection.

14. The cannabinoid measuring and detection device of claim 12, wherein the detection component and the output component are operably connected via a wireless connection.

15. The cannabinoid measuring and detection device of claim 11, wherein the output component further includes a display configured to provide an output indicative of whether the cannabinoid is present in the individual.

16. The cannabinoid measuring and detection device of claim 11, further comprising an output device, wherein the output device is configured to provide an output indicative of whether the cannabinoid is present in the individual.

17. The cannabinoid measuring and detection device of claim 11, further comprising an output device, wherein the output device is configured to provide an output indicative of a level of cannabinoid present in the individual.

18. A cannabinoid measuring and detection device, comprising:
a power source;
a UV light source operably connected to the power source and configured to output light in an output direction at an ultraviolet wavelength and at a known intensity;
a UV light sensor arranged in the output direction to detect the light output by the UV light source and output a detection signal corresponding to the detected light;
at least one processor operably connected to the UV light sensor and the UV light source;
a first arm in which the UV light source is arranged;
a second arm in which the UV light sensor is arranged; and
a connecting portion coupling the first arm with the second arm, the connecting portion enabling adjustment of a position of the first and second arm with respect to each other,
wherein the UV light source and UV light sensor are arranged to enable body tissue of an individual to be placed between the UV light source and UV light sensor,
wherein the at least one processor is configured to receive the detection signal corresponding to a test period during which the body tissue of the individual is placed between the UV light source and UV light sensor and determine whether a cannabinoid is present in the individual based on the known intensity of the output light and the detection signal,
wherein the UV light source outputs light with wavelengths between 240 and 370 nanometers, and
wherein, during the test period, the at least one processor determines whether the cannabinoid is present in the individual based on a difference between the known intensity of the output light and an intensity of the detected light represented by the detection signal.

19. The cannabinoid measuring and detection device of claim 18, wherein the difference between the known intensity of the output light and the intensity of the detected light represented by the detection signal is related to absorbance of the output light by the body tissue of the individual.

20. The cannabinoid measuring and detection device of claim 18, wherein the connecting portion enables adjustment of the position of the first and second arm while maintaining the UV light sensor in the output direction of the light output from UV light source.
